(19) United States
(12) Patent Application Publication
(10) Pub. No.: US 2015/0238429 A1
(43) Pub. Date: Aug. 27, 2015

(54) SOFT ELASTIC CAPSULES CONTAINING TABLETS AND LIQUID OR SEMISOLID FILLS AND METHODS FOR THEIR MANUFACTURE

(71) Applicant: BANNER LIFE SCIENCES LLC, High Point, NC (US)

(72) Inventors: Qi Fang, Oak Ridge, NC (US); Don A. Archibald, Jamestown, NC (US); Madhu Sudan Hariharan, Greensboro, NC (US); Roger E. Gordon, Stokesdale, NC (US)

(73) Assignee: BANNER LIFE SCIENCES LLC

(21) Appl. No.: 14/711,835

(22) Filed: May 14, 2015

Related U.S. Application Data
(62) Division of application No. 13/862,199, filed on Apr. 12, 2013.
(60) Provisional application No. 61/623,737, filed on Apr. 13, 2012.

(51) Int. Cl. A61K 9/48; A61K 31/616; A61K 31/4365; A61K 31/56; A61K 31/122; A61K 31/192; A61K 31/40; A61K 31/135; A61K 31/4545; A61K 31/80; A61K 31/451; A61K 31/137; A61K 45/06; A61K 31/155; A61K 31/4439; A61K 31/445; A61K 31/352; A61K 31/202

(52) U.S. Cl. CPC A61K 9/4808; A61K 31/202; A61K 31/616; A61K 31/4365; A61K 31/56; A61K 31/122; A61K 31/192; A61K 31/40; A61K 31/135; A61K 31/4545; A61K 31/80; A61K 31/451; A61K 31/137; A61K 45/06; A61K 31/155; A61K 31/4439; A61K 31/445; A61K 31/352; A61K 9/4825; A61K 9/4891; A61K 9/4833; A61K 9/4883

(57) ABSTRACT

Disclosed herein is a soft elastic capsule that includes an acid resistant, capsule shell that defines an encapsulated space having a predetermined volume, a liquid or semisolid fill comprising a first active ingredient located within the encapsulated space, and a first compressed tablet a having a minimal dimension of 2 mm, being located within the encapsulated space, unanchored to the capsule shell, and surrounded by the fill, said tablet comprising a second active ingredient that is substantially insoluble in the fill. A method of manufacturing a soft elastic capsule is also disclosed.

### CROSS REFERENCE TO RELATED APPLICATIONS

This application is a divisional of U.S. patent application Ser. No. 13/862,199, filed Apr. 12, 2013, which claims priority to U.S. Provisional Application No. 61/623,737, filed Apr. 13, 2012, both of which are incorporated herein by reference in their entirety.

### TECHNICAL FIELD

Disclosed herein is a soft elastic capsule that includes an acid resistant, capsule shell that defines an encapsulated space having a predetermined volume, a liquid or semisolid fill comprising a first active ingredient located within the encapsulated space, and a first compressed tablet a having a minimal dimension of 2 mm, being located within the encapsulated space, unanchored to the capsule shell, and surrounded by the fill, said tablet comprising a second active ingredient that is substantially insoluble in the fill. A method of manufacturing a soft elastic capsule is also disclosed.

### BACKGROUND

Combination therapies are employed in treating many types of diseases. In some instances, the dosage forms of two or more of the prescribed or desired active agents are incompatible with each other. Due to this limitation, the active agents are required to be administered separately, which can lead to poor compliance with treatment regimens due to the number of pills and liquids that must be consumed.

### SUMMARY

Described herein is a soft elastic capsule and a method for manufacturing the same. The soft elastic capsule can be an acid resistant, capsule shell that defines an encapsulated space having a predetermined volume, a liquid or semisolid fill comprising a first active ingredient located within the encapsulated space, and a first compressed tablet. The first compressed tablet is located within the encapsulated space, can be unanchored to the capsule shell, and can be surrounded by the fill. The tablet is substantially insoluble in the fill. The first compressed tablet can have a minimal dimension of 2 mm (e.g., a minimal dimension of 5 mm). In some embodiments, the first compressed tablet has a maximum dimension of 16 mm. The volume of the tablet can optionally be at least 25% smaller than the volume of the encapsulated space. The volume ratio of the first compressed tablet to the liquid or semisolid fill can be from 1:0.25 to 1:100. In some embodiments, the fill is liquid. In some embodiments, the capsule shell has only one compartment.

In some embodiments, the first active ingredient and/or the second active ingredient is a pharmaceutical, a nutraceutical, a vitamin, a mineral, or a diagnostic agent. For example, the first active ingredient can be a pharmaceutically active ingredient dissolved in a pharmaceutically acceptable oil-based liquid vehicle and the second active ingredient can be a pharmaceutically active ingredient. Optionally, the first active ingredient is different from the second active ingredient. In some embodiments, the first active ingredient is a polyunsaturated fatty acid (e.g., an omega-3 fatty acid) and the second active ingredient is acetylsalicylic acid, a statin (e.g., atorvastatin), clopidogrel, phytosterol, coenzyme Q10, resveratrol, bexarotene, or a combination of bexarotene and a statin.

In some embodiments, the first active ingredient is diphenhydramine and the second active ingredient is loratadine. In some embodiments, the first active ingredient is simethicone and the second active ingredient is loperamide. Optionally, the first active ingredient is an anti-allergy agent (e.g., cetirizine, loratadine, fexofenadine, diphenhydramine, levocetirizine, and desloratadine) and the second active ingredient is pseudoephedrine. Optionally, the first active ingredient is a serotonin 5-HT1A partial agonist or a selective serotonin re-uptake inhibitor and the second active ingredient is bupropion. In some embodiments, the first active ingredient is metformin and the second active ingredient is miglitol or pioglitazone. In some embodiments, the first active ingredient is lubiprostone and the second active ingredient is an opioid (e.g., oxycodone, hydrocodone, or morphine).

The capsule shell can be formed of a film-forming natural polymer. In some examples, the film-forming natural polymer can include gelatin. In some examples, the film-forming natural polymer can include carrageenan and/or starch. The film-forming natural polymer can be from about 20% to about 50% by weight of the capsule shell. Optionally, the capsule shell includes an enteric or gastric-resistant polymer. The capsule shell can further be formed of a gastric-resistant natural polymer, which can optionally include pectin and/or alginate. The concentration of the gastric-resistant natural polymer can be from about 2% to about 10% by weight of the capsule shell. The capsule shell can be further formed of a gelling agent. The gelling agent can include, for example, a divalent cation salt (e.g., calcium salts and/or magnesium salts). In some examples, the concentration of the gelling agent is less than about 2% by weight of the capsule shell. The capsule shell can be formed of a film-forming gastric-resistant synthetic polymer. In some examples, the film-forming gastric-resistant synthetic polymer can be selected from the group consisting of methacrylate, ethyl acrylate, cellulose acetate phthalate, polyvinyl acetate phthalate, or a combination of these. The capsule shell can further include one or more plasticizers selected from the group consisting of glycerin, sorbitol, sorbitan, maltitol, glycerol, polyethylene glycol, polyalcohols with 3 to 6 carbon atoms, citric acid, citric acid esters, triethyl citrate, and combinations thereof. In some examples, the concentration of the one or more plasticizers is from about 8% to about 40% by weight of the capsule shell.

Optionally, the soft elastic capsule can further comprise a second compressed tablet, which can optionally comprise a third active ingredient. In some embodiments, the second active ingredient is incompatible with the first active ingredient. The capsule shell can be transparent or translucent. In some embodiments, the compressed tablet is coated with a delayed-release coating or a sustained-release coating. Optionally, the soft elastic capsule comprises one or more pharmaceutically acceptable excipients.

Also described herein is a method of manufacturing a soft elastic capsule. The method comprises (a) forming a continuous first film comprising a film-forming polymer on a first rotating encapsulation die, (b) forming a continuous second film comprising a film-forming polymer on a second rotating encapsulation die, (c) rotating the first rotating encapsulation die and the second rotating encapsulation die in counter directions to contact the first film and second film and form a partially closed capsule, (d) providing a first compressed tablet in the partially closed capsule, (e) injecting a liquid or semisolid fill into the partially closed capsule, (f) sealing the partially closed capsule to form a soft capsule, and (g) drying and finishing the soft capsule. In some embodiments, the providing step (d) comprises (d1) positioning a first compressed tablet onto the first film on the first encapsulation die, and (d2) optionally positioning a second compressed tablet onto the second film on the second encapsulation die. In these embodiments, said step (c) of rotating the first rotating encapsulation die and the second rotating encapsulation die in counter directions to contact the first film and second film and form a partially closed capsule provides the first compressed tablet and the optional second compressed tablet in the partially closed capsule. Step (d1) can comprise positioning a first compressed tablet onto the first film and into a die cavity on the first encapsulation die and step (d2) can comprise optionally positioning a second compressed tablet onto the second film and into a die cavity on the second encapsulation die. The providing step (d) can comprise feeding the first compressed tablet and optionally a second compressed tablet into the partially closed capsule after said rotating step (c).

The method can further comprise providing an encapsulation wedge adjacent the location where the first film and second film are contacted. In some embodiments, the first compressed tablet is fed through the encapsulation wedge. In some embodiments, the liquid or semisolid fill is injected through the encapsulation wedge. Optionally, the encapsulation wedge is heated. In some embodiments, the first compressed tablet and the optional second compressed tablet are pre-manufactured.

In some embodiments, the film-forming polymer in the first film and the second film is a natural film-forming polymer. Optionally, the natural film-forming polymer in the first film and the second film comprises gelatin. In some examples, the steps of forming the first film and forming the second film comprise preparing a solution comprising gelatin, a gastric-resistant natural polymer, and optionally one or more plasticizers to form a gel mass; and forming the gel mass into the first film and the second film. Optionally, the gastric-resistant natural polymer includes pectin and/or alginate. In some embodiments, the liquid or semisolid fill comprises a first active ingredient, the first compressed tablet comprises a second active ingredient, and the second compressed tablet comprises a third active ingredient. The first active ingredient, the second active ingredient, and the third active ingredient can be different from each other.

The details of one or more embodiments are set forth in the description below and in the drawings. Other features, objects, and advantages will be apparent from the description, the drawings, and from the claims.

### BRIEF DESCRIPTION OF THE DRAWINGS

FIG. 1A is a schematic depicting a soft elastic capsule containing a liquid or semisolid fill and a single tablet.

FIG. 1B is a schematic depicting a soft elastic capsule containing a liquid or semisolid fill and multiple tablets.

FIG. 2 is a schematic of a rotary die process for manufacturing soft elastic capsules containing a liquid or semisolid fill and one tablet. An enlarged tablet is depicted in the bottom right corner.

FIG. 3 is a schematic of a rotary die process for manufacturing soft elastic capsules containing a liquid or semisolid fill and two tablets. An enlarged tablet is depicted in the bottom right corner.

FIG. 4 is a schematic of an alternative rotary die process for manufacturing soft elastic capsules containing a liquid or semisolid fill and one tablet provided from a hopper located distal to the liquid or semisolid fill injector. An enlarged tablet is depicted in the bottom right corner.

FIG. 5 is a schematic of an alternative rotary die process for manufacturing soft elastic capsules containing a liquid or semisolid fill and two tablets provided from hoppers located distal to the liquid or semisolid fill injector. An enlarged tablet is depicted in the bottom right corner.

FIG. 6 is a schematic of an alternative rotary die process for manufacturing soft elastic capsules containing a liquid or semisolid fill and one tablet provided from a hopper adjacent the encapsulation wedge.

### DETAILED DESCRIPTION

Soft elastic capsules and methods for their manufacture are disclosed herein. The soft elastic capsules can include an acid resistant capsule shell that defines an encapsulated space having a predetermined volume, a liquid or semisolid fill comprising a first active ingredient located within the encapsulated space, and a first compressed tablet having a minimal dimension of 2 mm, being located within the encapsulated space, unanchored to the capsule shell, and surrounded by the fill. The tablet is substantially insoluble in the fill.

The soft elastic capsules disclosed herein include dosage forms of active ingredients and excipients encapsulated by a film-forming composition (i.e., a capsule shell). As described above, the capsule shell defines an encapsulated space having a predetermined volume. The capsule shell of the soft elastic capsule disclosed herein can be acid resistant. As used herein, “acid resistant” refers to the enteric property of the capsule. Specifically, the capsule is resistant to dissolution in stomach acid for a period of time and can therefore pass through the stomach without substantial release of the active ingredients inside the capsule. As used herein, “substantial release” refers to a release of greater than 1% of the active ingredient (e.g., 1% to 100%, 5% to 95%, 10% to 90%, 20% to 80%, 30% to 70%, or 40% to 60% of the active ingredient). In some embodiments, the capsule is resistant to dissolution in stomach acid for at least 30 minutes. For example, the capsule can be resistant to dissolution in stomach acid for at least 40 minutes, at least 45 minutes, at least 50 minutes, at least 55 minutes, at least 1 hour, or at least 2 hours.

The capsule shell can include one or more layers and can be formed from one or more components, including film-forming polymers, gastric-resistant polymers, gelling agents, and plasticizers. The film-forming polymers can be film-forming natural polymers, film-forming synthetic polymers, film-forming semi-synthetic polymers, or mixtures thereof. In some embodiments, the capsule shell described herein includes at least one film-forming natural polymer, such as gelatin, carrageenans (e.g., kappa carrageenan, iota carrageenan, and blends of these), glucomannans, starches (e.g., unmodified starch and modified pregelatinized starch), other hydrocolloids, and mixtures of these. In some embodiments, the capsule shell described herein includes at least one film-forming gastric-resistant synthetic polymer such as a methacrylate polymer, an ethyl acrylate polymer, an acrylic polymer (e.g., EUDRAGIT® acrylic polymer provided by Evonik Industries; Parsippany, N.J.), cellulose acetate phthalate, or polyvinyl acetate phthalate. The film-forming polymer can be from about 20% to about 50% by weight of the capsule shell. For example, the film-forming polymer can be from about 25% to about 40% by weight of the capsule shell. In some examples, the film-forming polymer is about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, or about 49% by weight of the capsule shell.

As described above, the capsule shell can include gelatin. The gelatin suitable for use herein can be derived from pork skins, pork and cattle bones, or split cattle hides. Gelatin is a mixture of peptides and proteins produced by partial hydrolysis of collagen extracted from the skin, boiled crushed bones, connective tissues, organs, and some intestines of animals, including, for example, domesticated cattle, chicken, and pigs.

In many respects, the chemical composition of gelatin is similar to that of collagen. The natural molecular bonds between individual partially hydrolyzed collagen strands in gelatin are broken down into a form that can rearrange. Gelatin can melt to a liquid when heated and can re-solidify upon cooling. Gelatin forms a high viscosity solution in hot water, which sets upon cooling to form a semi-solid colloid gel.

Gelatin solutions show viscoelastic flow and streaming birefringence. As described above, gelatin can swell and form a semi-solid material in the presence of cold water. However, gelatin is readily soluble in hot water. The solubility of the gelatin is determined by the method of manufacture. Gelatin can be dispersed in a relatively concentrated acid. Such dispersions can be stable for several days (e.g., up to 15 days) with little or no chemical changes and are suitable for coating purposes or for extrusion into a precipitating bath. The capsule shell can further include one or more gastric-resistant polymers such as gastric-resistant natural polymers, gastric-resistant synthetic polymers, or mixtures thereof, to provide enteric properties to the capsule shell. Thus, as described above, the soft elastic capsules, when ingested, will pass through the stomach without dissolving. In some embodiments, the film-forming polymer can include gastric-resistant properties. The capsule shell can include a single layer formed from a composition that includes the gastric-resistant polymer or can include multiple layers including at least an outside coating layer that includes the gastric-resistant polymer. In some embodiments, the capsule shell can further be formed of a gastric-resistant natural polymer, including a polysaccharide such as pectin and/or alginates. The gastric-resistant polymer can be included in the capsule shell in an amount of from about 2% to about 10% by weight of the capsule shell.

The capsule shell of the soft elastic capsule can further be formed of one or more gelling agents. The gelling agent can include, for example, one or more divalent cations. The divalent cations can be provided as divalent cation salts (e.g., calcium salts and magnesium salts). When included, the concentration of the gelling agent can be greater than 0% by weight and less than about 2% by weight of the capsule shell (e.g., less than about 1.5%, less than about 1.0%, or less than about 0.5% by weight of the capsule shell).

In some embodiments, the capsule shell can include one or more plasticizers. The plasticizer can be, for example, glycerol. Glycerol (i.e., glycerine or glycerin) is a colorless, odorless, viscous liquid that is widely used in pharmaceutical formulations. Glycerol is a polyol containing three hydroxyl groups that are responsible for its solubility in water and its hygroscopic nature. Glycerol is sweet tasting and has low toxicity. For human consumption, glycerol is classified by the U.S. FDA among the sugar alcohols as a caloric macronutrient.

In some embodiments, the plasticizer is sorbitol. Sorbitol is a sugar alcohol that the human body metabolizes slowly. It can be obtained by reducing the aldehyde group of glucose to a hydroxyl group. Sorbitol is naturally found in apples, pears, peaches, and prunes. A special grade of aqueous sorbitol solution is used in softgel capsules as a plasticizer to prevent capsules from becoming brittle. In some embodiments, sorbitol is included in capsule shells that will contain polyethylene glycol as a solvent. Further examples of suitable plasticizers include sorbitan, maltitol, polyethylene glycol, polyalcohols with 3 to 6 carbon atoms, citric acid, citric acid esters, triethyl citrate, and combinations of these. The concentration of the one or more plasticizers can be from about 8% by weight to about 40% by weight of the capsule shell. In some examples, the concentration of the plasticizers is from about 10% by weight to about 30% by weight or from about 15% by weight to about 25% by weight of the capsule shell.

Optionally, the capsule shells can include one or more viscosity modifiers. Examples of suitable viscosity modifiers include guar gum, locust bean gum, xanthan gum, agar, and gellan gum. The viscosity modifier can be included in the capsule shell in an amount of greater than 0% by weight and less than 10% by weight of the composition (e.g., less than 9%, less than 8%, less than 7%, less than 6%, less than 5%, less than 4%, less than 3%, less than 2%, less than 1%, or less than 0.5% by weight of the composition).

Optionally, the capsule shell can be prepared as a transparent or translucent capsule shell. In some embodiments, the capsule shell can be semi-transparent, semi-opaque, or opaque. Optionally, the opaque capsule shells are prepared using titanium dioxide, which can protect light sensitive active ingredients from degradation. The capsule shells can further include a colorant to color the capsules. Examples of suitable colorants include FD&C and D&C dyes, iron oxides, and natural colorants. Optionally, the capsule can be imprinted or have a decorative coating. The capsule shell can be prepared to have only one compartment (i.e., the capsule shell does not contain multiple compartments).

Combinations of at least two active ingredients and, optionally, excipients are contained within the capsule shell. The capsules are multi-phasic in that the capsules contain two or more phases of matter. For example, the capsules can include active ingredients in a solid phase, a semisolid phase, and/or a liquid phase. The soft elastic capsules disclosed herein include a liquid or semisolid fill located within the encapsulated space. The liquid or semisolid fill includes a first active ingredient. The first active ingredient can include, for example, a pharmaceutical, a nutraceutical, a vitamin, a mineral, or a diagnostic agent.

Examples of pharmaceutical agents that can be included as an active ingredient include agents classified as, for example, an adrenocortical steroid, adrenocortical suppressant, aldosterone antagonist, amino acid, anabolic, androgen, antagonist, anthelmintic, anti-acne agent, anti-adrenergic, anti-allergic, anti-amebic, anti-androgen, anti-anemic, anti-anginal, anti-arthritic, anti-asthmatic, anti-atherosclerotic, antibacterial, anticholelithic, anticholelithogenic, anticholinergic, anticoagulant, anticoccidal, antidiabetic, antidiarrheal, antidiuretic, antidote, anti-estrogen, antifibrinolytic, antifungal, antiglaucoma agent, antihemophilic, antihemorrhagic, antihistamine, antihyperlipidemia, antihyperlipoproteinemic, antihypertensive, antihypotensive, anti-infective, anti-infective, topical, anti-inflammatory, antikeratinizing agent, antimalarial, antimicrobial, antimitotic, antimycotic, antineoplastic, antineutropenic, antiparasitic, antiperistaltic, antipneumocystic, antiproliferative, antiprostatic hypertrophy, antiprotozoal, antipruritic, antipsoriatic, antirheumatic, antischistosomal, antiseborrheic, antisecretory, antispasmodic, antithrombotic, antitussive, anti-ulcerative, anti-urolithic, antiviral, appetite suppressant, benign prostatic hyperplasia therapy agent, bone resorption inhibitor, bronchodilator, carbonic anhydrase inhibitor, cardiac depressant, cardioprotectant, cardiotonic, cardiovascular agent, choleretic, cholinergic, cholinergic agonist, cholinesterase deactivator, coccidiostat, diagnostic aid, diuretic, ectoparasiticide, enzyme inhibitor, estrogen, fibrinolytic, free oxygen radical scavenger, glucocorticoid, gonad-stimulating principle, hair growth stimulant, hemostatic, hormone, hypocholesterolemic, hypoglycemic, hypolipidemic, hypotensive, immunizing agent, immunomodulator, immunoregulator, immunostimulant, immunosuppressant, impotence therapy adjunct, inhibitor, keratolytic, LHRH agonist, liver disorder treatment, luteolysin, mucolytic, mydriatic, nasal decongestant, neuromuscular blocking agent, non-hormonal sterol derivative, oxytocic, plasminogen activator, platelet activating factor antagonist, platelet aggregation inhibitor, potentiator, progestin, prostaglandin, prostate growth inhibitor, prothyrotropin, pulmonary surface, radioactive agent, regulator, relaxant, repartitioning agent, scabicide, sclerosing agent, selective adenosine A1 antagonist, steroid, suppressant, symptomatic multiple sclerosis, synergist, thyroid hormone, thyroid inhibitor, thyromimetic, amyotrophic lateral sclerosis agents, Paget's disease agents, unstable angina agents, uricosuric, vasoconstrictor, vasodilator, vulnerary, wound healing agent, and xanthine oxidase inhibitor. Further examples of suitable pharmaceutical agents include those as listed in the Merck Index (13^{th} Edition, Wiley, 2001), The United States Pharmacopeia-National Formulary (USP-NF), and the FDA's Orange book, which are each incorporated by reference herein at least for their teachings of pharmaceutically active agents.

Examples of nutraceuticals include, but are not limited to, amino acids, terpenoids (e.g., carotenoid terpenoids and non-carotenoid terpenoids), herbal supplements, homeopathic supplements, glandular supplements, polyphenolics, flavonoid polyphenolics, phenolic acids, curcumin, resveratrol, lignans, glucosinolates, isothiocyanates, indoles, thiosulfonates, phytosterols, anthraquinones, capsaicin, piperine, chlorophyll, betaine, oxalic acid, acetyl-L-carnitine, allantoin, androsterondiol, androsterondione, betaine (trimethylglycine), caffeine, calcium pyruvate (pyruvic acid), carnitine, carnosine, carotene, carotenoid, choline, chlorogenic acid, cholic acid, chondroitin sulfate, chondroitin sulfate, cholestan, chrysin, coenzyme Q10, conjugated linoleic acid, corosolic acid, creatine, dehydroepiandrosterone, dichlorophen, diindolymethane, dimethylglycine, dimercapto succinic Acid, ebselen, ellagic acid, enzymes, fisetin, formonetin, glucaric acid (glucarate), glucosamine (HCl or sulfate), glucosamine (N-acetyl), glutathione, hesperidine, hydroxy-3-methylbutyric Acid, 5-hydroxytryptophan, indole-3-carbinol, inositol, isothiocyanates, linolenic acid-gamma, lipoic acid (alpha), melatonin, methylsulfonylmethane, minerals, naringin, pancreatin, para-aminobenzoic acid, paraben (methyl or propyl), phenolics, phosphatidylcholine, phosphatidylserine, phospholipids, phytosterols, progesterone, pregnenolone, quercetin, resveratrol, D-ribose, rutin, S-adenosylmethionine, salicylic acid, sulforaphane, tartaric acid, taxifolin, tetrahydropalmatine, theophyline, theobromine, tigogenin, troxerutin, tryptophan, tocotrienol (alpha, beta & gamma), zeaxanthin, gingko biloba, ginger, cat's claw, hypericum, aloe vera, evening primrose, garlic, capsicum, dong quai, ginseng, feverview, fenugreek, echinacea, green tea, marshmallow, saw palmetto, tea tree oil, fish oil, payllium, kava-kava, licorice root, mahonia aquifolium, hawthorne, yohimbe, tumeric, witch Hazel, valerian, mistletoe, bilberry, bee pollen, peppermint oil, beta-carotene, genistein, lutein, lycopene, the polyphenols, and the like. Further examples of suitable nutraceuticals include those listed in Handbook of Nutraceuticals and Functional Foods, edited by Robert E. C. Wildman, CRC Press (2001), which is incorporated by reference herein at least for their teachings of nutraceuticals.

In some embodiments, the first active ingredient can include one or more vitamins. As used herein, vitamin refers to any organic substance that is typically essential for the normal growth and activity of humans. Examples of suitable vitamins include, but are not limited to, vitamin A (retinol), B1 (thiamine), B2 (riboflavin), B complex, B6 (pyridoxine), B12 (cobalamin), C (ascorbic acid), D (cholecalciferol), E (tocopherol), F (linoleic acid), G, H (biotin), and K, and choline, folic acid, inositol, niacin, pantothenic acid, and para-aminobenzoic acid.

Minerals are naturally occurring inorganic substances which are typically essential to the nutrition of humans. The mineral for use in the first active ingredient can be any mineral. Examples of minerals include, but are not limited to, boron, calcium, chromium, copper, iron, magnesium, manganese, molybdenum, nickel, phosphorus, selenium, silicon, tin, vanadium, and zinc.

The first active ingredient can optionally include a diagnostic agent. Diagnostic agents include, for example, imaging agents, contrasting agents, enzymes, and fluorescent substances.

In some embodiments, the first active ingredient is innately a liquid or semisolid. In other embodiments, the first active ingredient can be prepared as a liquid. Liquid active ingredients can, for example, be prepared by dissolving or otherwise mixing an active compound as defined above and optional pharmaceutical adjuvants in a carrier, such as, for example, water, saline, aqueous dextrose, glycerol, glycols (e.g., propylene glycol or polyethylene glycol), ethanol, fatty acids, glycerides, oils, sterols, phospholipids, and the like, to thereby form a solution. In some embodiments, the active ingredient can be dispersed or suspended in the liquid carrier. In some embodiments, the first active ingredients can be prepared in a self-emulsifying/microemulsifying drug delivery system (SEDDS) system. Optionally, the SEDDS system can include an oil, a surfactant, a cosurfactant or solubilizer, and the first active ingredient. Actual methods of preparing such dosage forms are known, or will be apparent, to those skilled in this art; for example, see Remington's Pharmaceutical Sciences, Mack Publishing Company, Easton, Pa., 15th Edition, 1975. The liquid active ingredients can be prepared to contain the active ingredient in the range of 0.005% to 100%, with the balance made up from non-toxic carrier. Methods for preparation of these compositions are known to those skilled in the art. The liquid fill can contain 0.001% to 100%, 0.1% to 95%, 1% to 90%, 5% to 70%, or 10% to 50% by weight active ingredient.

One or more compressed tablets are also located within the encapsulated space formed by the capsule shell. The one or more compressed tablets are substantially insoluble in the fill. “Substantially insoluble” tablets include tablets having very low chemical solubility in the fill (e.g., less than 10 g of the tablet can dissolve in 100 ml of the fill). Therefore, the tablets can retain the compressed tablet form after a period of time. For example, a substantially insoluble tablet can retain at least 90%, at least 95%, or at least 99% of its form in the fill after a period of one year. The compressed tablets present within the encapsulated space can be unanchored to the capsule shell. As used herein, “unanchored” refers to the compressed tablets being unattached to the capsule shell and thus capable of free movement throughout the encapsulated space. The volume of the tablet is smaller than the volume of the encapsulated space. Thus, the compressed tablets can be surrounded by the fill (i.e., the fill can be present on all sides of compressed tablets and in contact with the outer surface of the compressed tablet). In some embodiments, the volume of the tablet is at least 25% smaller than the volume of the encapsulated space. For example, the volume of the tablet can be at least 30%, at least 35%, at least 40%, at least 45%, or at least 50% smaller than the volume of the encapsulated space. In some embodiments, the volume ratio of the compressed tablet to the liquid or semisolid fill is from 1:0.25 to 1:100 (e.g., from 1:1 to 1:75 or from 1:5 to 1:50). In some embodiments, the weight ratio of the compressed tablet to the liquid or semisolid fill is from 1:5 to 1:100 (e.g., from 1:10 to 1:75 or from 1:25 to 1:50). The outer surface of the compressed tablet can optionally be coated with a delayed-release coating or a sustained-release coating.

Optionally, the compressed tablet is a membrane-controlled release tablet such as an Oros system tablet commercially available from Alza Corporation (Mountain View, Calif.). In some examples, the compressed tablet is a matrix-type controlled release tablet. In these examples, the compressed tablet can include release rate controlling excipients within the tablet matrix. As used herein,” release rate controlling excipient” includes any substance capable of slowing the release rate of the active ingredient from the compressed tablet dosage form. The release rate controlling excipient can be, for example, a polymer, a fatty compound, or a mixture of these. Suitable polymeric release rate controlling excipients include, for example, cellulose ethers (e.g., methylcellulose, ethylcellulose, hydroxyethylcellulose, hydroxypropyl cellulose, hydroxypropyl methylcellulose, hydroxypropyl ethylcellulose, carboxymethyl cellulose, crosslinked carboxymethyl cellulose and its alkali salts, ethyl hydroxyethylcellulose, hydroxyethyl methylcellulose, hydrophobically modified hydroxyethyl cellulose, hydrophobically modified ethyl hydroxyethylcellulose, carboxymethyl hydroxyethylcellulose, and carboxymethyl hydrophobically modified hydroxyethyl cellulose); vinyl pyrrolidone polymers (e.g., crosslinked polyvinylpyrrolidone or copolymers of vinyl pyrrolidone and vinyl acetate); alkylene oxide homopolymers (e.g., polypropylene oxide); super disintegrant polymers (e.g., crosslinked polyvinylpyrrolidone, cross-linked sodium carboxymethylcellulose, carboxymethyl starch, sodium carboxymethyl starch, potassium methacrylate-divinylbenzene copolymer, polyvinyl alcohols, amylose, cross-linked amylose, starch derivatives, microcrystalline cellulose and cellulose derivatives, alpha-, beta- and gamma-cyclodextrin and dextrin derivatives such as cross-linked carboxymethylcellulose); gums of plant, animal, mineral or synthetic origin (e.g., agar, alginates, carrageenan, furcellaran derived from marine plants, guar gum, gum arabic, gum tragacanth, karaya gum, locust bean gum, pectin derived from terrestrial plants); microbial polysaccharides (e.g., dextran, gellan gum, rhamsan gum, welan gum, xanthan gum), synthetic or semi-synthetic gums (e.g., propylene glycol alginate, hydroxypropyl guar and modified starches such as sodium starch glycolate); and acrylic acid polymers such as cross-linked polymers or homopolymers and co-polymers of acrylate or methacrylate monomers. Suitable fatty compounds for use as the release rate controlling excipients include waxes (e.g., digestible, long chain (C₈-C₅₀, especially C₁₂-C₄₀)), substituted or unsubstituted hydrocarbons (e.g., fatty acids, fatty alcohols, glyceryl esters of fatty acids), and mineral and vegetable oils.

The tablets as described herein can be prepared using techniques and procedures known to those of skill in this art; see, for example, Ansel Introduction to Pharmaceutical Dosage Forms, Seventh Edition, 1999. The tablets can be made, for example, by direct compression, dry granulation (e.g., by slugging or roller compaction), or wet granulation. Direct compression involves blending the ingredients in a blender or mixer and compressing the ingredients of the tablet directly without changing the physical and chemical properties of the active ingredients. Dry granulation can include the steps of blending the ingredients, slugging the ingredients, dry screening, lubrication, and compression. The wet granulation method can include mixing the ingredients in a suitable blender, followed by adding a granulating solution under shear (e.g., low shear or high shear) to obtain a granulation. The damp mass can then be screened through a suitable screen and dried by tray drying or fluidized bed drying. Optionally, the wet mass can be dried and passed through a mill.

As understood by those of skill in the art, tablets can have a variety of shapes. For example, the tablets can be round, ovoid, polygonal, or polyhedral (e.g., triangular or rectangular). Thus, as used herein, dimension refers to the distance between two antipodal points of the tablet. For example, dimension can refer to diameter in a round tablet. In some examples, the compressed tablets have a minimal (i.e., minimum) dimension of 2 mm or greater. For example, the compressed tablets can have a minimal dimension of 3 mm, 4 mm, 5 mm, 6 mm, 7 mm, 8 mm, 9 mm, or 10 mm. In some examples, the compressed tablets can have a maximum dimension of 16 mm or less. For example, the compressed tablets can have a maximum dimension of 15 mm, 14 mm, 13 mm, 12 mm, 11 mm, or 10 mm. In some examples, the tablets can have convex or concave surfaces. In some examples, the tablets can contain one or more cavities (e.g., a recess or a hole).

The encapsulated space includes at least one compressed tablet. A first compressed tablet is located within the encapsulated space that includes a second active ingredient. The second active ingredient is a pharmaceutical, a nutraceutical, a vitamin, a mineral, or a diagnostic agent. Suitable pharmaceutical agents, nutraceuticals, vitamins, minerals, and diagnostic agents include those as described herein. Optionally, the first active ingredient and the second active ingredient are the same to provide different release profiles. Optionally, the first active ingredient is different from the second active ingredient. In some embodiments, the second active ingredient is incompatible with the first active ingredient. Optionally, a second compressed tablet can be located within the second compressed tablet. The second compressed tablet can include a third active ingredient such as a pharmaceutical, a nutraceutical, a vitamin, a mineral, or a diagnostic agent as described herein.

One or more pharmaceutically acceptable excipients can further be encapsulated within the capsule shell. Examples of pharmaceutically acceptable excipients include buffers, such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers, such as polyvinyl pyrrolidone; amino acids such as glycine, glutamine, asparagine, arginine or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN® (ICI, Inc.; Bridgewater, N.J.), polyethylene glycol (PEG), and PLURONICS™ (BASF; Florham Park, N.J.). Diluents commonly used in the art can also be encapsulated within the shell, including water or other solvents, solubilizing agents, and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, sesame oil, glycerol, tetrahydrofurfuryl alcohol, polyethylene glycols, and fatty acid esters of sorbitan, or mixtures of these substances, and the like.

Specific combinations of first active ingredients and second active ingredients are contemplated herein. In some embodiments, the first active ingredient is a pharmaceutically active ingredient dissolved in a pharmaceutically acceptable oil-based liquid vehicle and the second active ingredient is a pharmaceutically active ingredient. Optionally, the first and second active ingredients are combined in a manner to provide a combination therapy for treating a specific disease, illness, condition, or ailment. For example, a polyunsaturated fatty acid, such as an omega-3 fatty acid, can be provided as a first active ingredient and acetylsalicylic acid (i.e., aspirin) can be provided as a second active ingredient to treat or prevent cardiovascular conditions. In some examples, the aspirin can include an enteric coating. Combining these two agents into a single dosage form as described herein provides the advantages of preventing the side effect of “fishy” smells from the omega-3 fatty acids, preventing irritation to gastric mucosa by aspirin, and avoiding the hydrolysis of aspirin by preventing the direct contact of aspirin and water. A further combination of agents suitable for treating or preventing cardiovascular conditions includes a polyunsaturated fatty acid and a statin (e.g., atorvastatin) as the first and second active ingredients, respectively. In some examples, a combination of agents suitable for treating or preventing cardiovascular conditions includes a polyunsaturated fatty acid as the first active ingredient and clopidogrel as the second active ingredient. In other examples, a combination of agents includes a polyunsaturated fatty acid as the first active ingredient and one or more of phytosterol, coenzyme Q10, or resveratrol as the second active ingredient. A combination of agents can further include a polyunsaturated fatty acid as the first active ingredient and bexarotene, a statin, or a combination of bexarotene and a statin as the second active ingredient, and can be used to treat age related dementia (e.g., Alzheimer's disease).

Optionally, the first active ingredient is diphenhydramine and the second active ingredient is loratadine. In some examples, the first active ingredient is simethicone and the second active ingredient is loperamide. In some embodiments, the first active ingredient can be an anti-allergic (i.e., anti-allergy agent) and the second active ingredient can be pseudoephedrine. The pseudoephedrine can be, for example, in an immediate release form or a controlled release form. Exemplary anti-allergy agents include cetirizine, loratadine, fexofenadine, diphenhydramine, levocetirizine, and desloratadine. A soft elastic capsule containing a serotonin 5-HT1A partial agonist or a selective serotonin re-uptake inhibitor as the first active ingredient and bupropion as the second active ingredient can be used, for example, to treat depressive disorders. A soft elastic capsule including metformin as the first active ingredient and miglitol or pioglitazone as the second active ingredient can be used to improve glycemic control of subjects.

In some examples, the soft elastic capsule can include lubiprostone as the first active ingredient and an opioid as the second active ingredient. Examples of suitable opioids include, for example, oxycodone, hydrocodone, or morphine. Soft elastic capsules including these combinations of active ingredients can be used to treat, for example, irritable bowel syndrome and constipation. In some embodiments, the opioid is oxycodone.

Also described herein is a method of manufacturing a soft elastic capsule. The method includes the steps of (a) forming a continuous first film comprising a film-forming polymer on a first rotating encapsulation die; (b) forming a continuous second film comprising a film-forming polymer on a second rotating encapsulation die; (c) rotating the first rotating encapsulation die and the second rotating encapsulation die in counter directions to contact the first film and second film and form a partially closed capsule; (d) providing a first compressed tablet in the partially closed capsule; (e) injecting a liquid or semisolid fill into the partially closed capsule; (f) sealing the partially closed capsule to form a soft capsule; and (g) drying and finishing the soft capsule.

The continuous first film and continuous second film can be prepared by combining and mixing ingredients used to form a capsule shell, as disclosed herein. The continuous first and second films can include film-forming polymers, gastric-resistant polymers, gelling agents, and plasticizers. In some examples, the film-forming polymer in the first film and the second film is a natural film-forming polymer as described herein. Optionally, the first film and the second film include gelatin. In these embodiments, the first film and second film are formed by preparing a solution comprising gelatin, a gastric-resistant natural polymer, and optionally one or more plasticizers to form a gel mass, and forming the gel mass into the first film and the second film. The gastric-resistant polymer can include, for example, a gastric-resistant natural polymer such as pectin or alginate or a gastric-resistant synthetic polymer such as a methacrylate polymer, an ethyl acrylate polymer, an acrylic polymer, cellulose acetate phthalate, or polyvinyl acetate phthalate. Optionally, the first film and second film can be cast individually on separate rotating casting drums in a continuous manner by introducing the gel mass to an outer casting surface of each drum. The cooling drums can be cooled to a temperature lower than the gel mass, which can cause the gel mass to solidify on the drum casting surface to form the films. The gel mass can be dispensed in a layer in an amount sufficient to provide the desired thickness of the film. The thickness of the film can range, for example, from 0.005 inches to 0.045 inches. The first and second films can then be fed from the casting drums to the first and second rotating encapsulation dies according to the methods described in U.S. Pat. No. 6,482,516, which is incorporated herein by reference in its entirety.

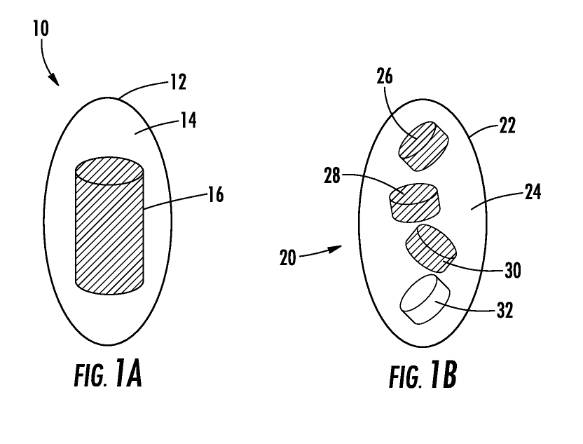

FIGS. 1A and 1B illustrate soft elastic capsules as described herein. FIG. 1A illustrates an embodiment where the soft elastic capsule 10 includes a capsule shell 12, such as an acid resistant capsule shell, and a liquid or semi-solid fill 14 that surrounds a tablet 16. FIG. 1B illustrates another embodiment where the soft elastic capsule 20 includes a capsule shell 22, such as an acid resistant capsule shell, and a liquid or semisolid fill 24 that surrounds multiple tablets 26, 28, 30, and 32.

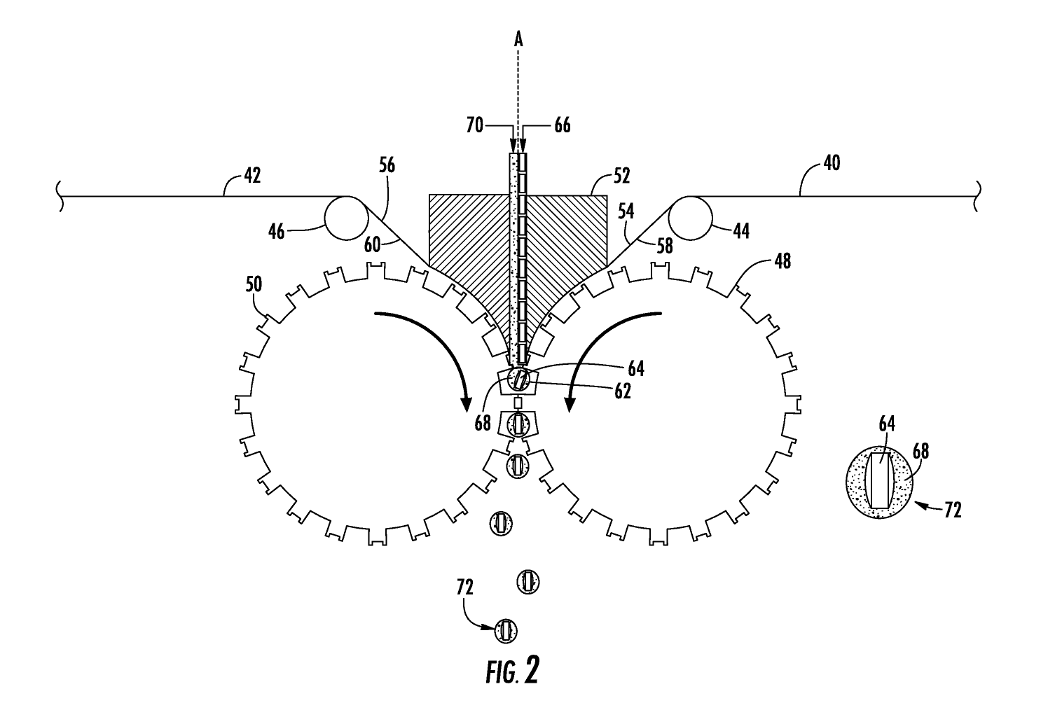

FIG. 2 illustrates an embodiment for making the soft elastic capsules described herein. As shown in FIG. 2, a continuous film 40 and a continuous film 42 can be fed over tractor rolls 44 and 46, respectively. The tractor rolls 44 and 46 optionally can be coated with a lubricant. The continuous film 40 advances from the tractor roll 44 to a rotating encapsulation die 48. Likewise, the continuous film 42 advances from the tractor roll 46 to a rotating encapsulation die 50. The encapsulation dies 48 and 50 rotate in opposite directions. For example, as shown in FIG. 2, the encapsulation die 48 can rotate counterclockwise while the encapsulation die 50 can rotate clockwise. A layer of lubricant from the tractor rolls 44 and 46 can be retained on the reverse surface of the films 40 and 42, respectively, to prevent the encapsulation dies 48 and 50 from sticking to the films 40 and 42 upon contact. Optionally, an encapsulation wedge 52 can be provided adjacent the location where the films 40 and 42 contact the encapsulation dies 48 and 50 as further described herein. In some embodiments, the encapsulation wedge 52 can be heated. The first and second encapsulation dies 48 and 50, together with an encapsulation wedge 52, can be symmetrically disposed relative to each other about a center plane A of the apparatus. The films 40 and 42 are advanced between the encapsulation wedge 52 and the encapsulation dies 48 and 50, respectively. In some embodiments, a surface 54 of the film 40 and a surface 56 of the film 42 contact the encapsulation wedge 52 and a reverse surface 58 of the first film 40 and a reverse surface 60 of the film 42 contact the encapsulation dies 48 and 50, respectively.

The rotating encapsulation die 48 and the rotating encapsulation die 50 rotate to advance the film 40 and the film 42 together to form a partially closed capsule 62. A compressed tablet 64 can be released from a tablet hopper 66 into the partially closed capsule 62. The compressed tablet 64 can be pre-manufactured (i.e., compressed and formed into a tablet prior to being provided in the tablet hopper 66). Optionally, as shown in FIG. 2, the tablet hopper 66 is provided in the encapsulation wedge 52 and the tablet 64 is released from the tablet hopper into the partially closed capsule 62. A liquid or semisolid fill 68 can be injected from a pump through a channel 70 into the partially closed capsule 62, for example, after the tablet 64 has been provided in the partially closed capsule. The channel 70 can be provided in the encapsulation wedge 52. Once the tablet 64 and the liquid or semisolid fill 68 are provided in the partially closed capsule 62, the partially closed capsule can be sealed to form a soft elastic capsule 72 as described herein. The soft elastic capsule can then be dried and finished according to methods known in the art.

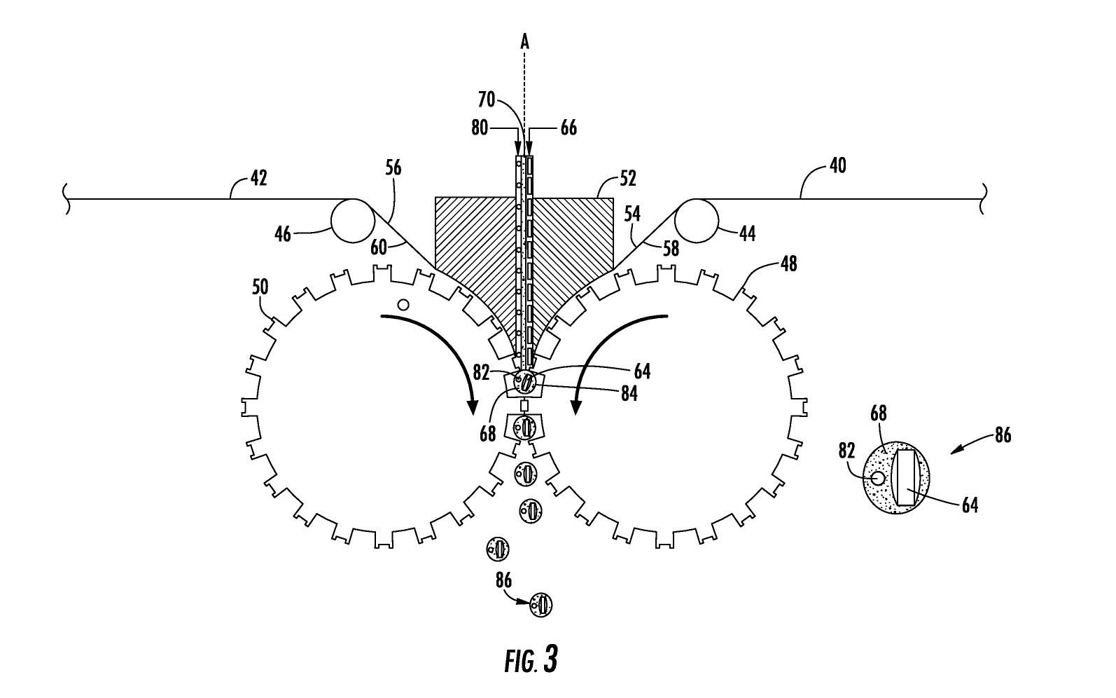

In some embodiments, the soft elastic capsule can include more than one tablet. FIG. 3 is similar to FIG. 2 but includes a second tablet hopper 80 for releasing a second compressed tablet 82 into a partially closed capsule 84. The second compressed tablet 82 can be pre-manufactured and can be released into the partially closed capsule 84 simultaneously with, before, or after the liquid or semisolid fill 68 and the compressed tablet 64. The partially closed capsule 84 can then be sealed as described above to form a soft capsule 86 containing multiple tablets 64 and 82. Although the process of FIG. 3 provided one method of providing multiple tablets in the same soft elastic capsule, other means can also be used. For example, more than one tablet can be released from the tablet hopper 66 illustrated in FIG. 2 (or FIG. 3).

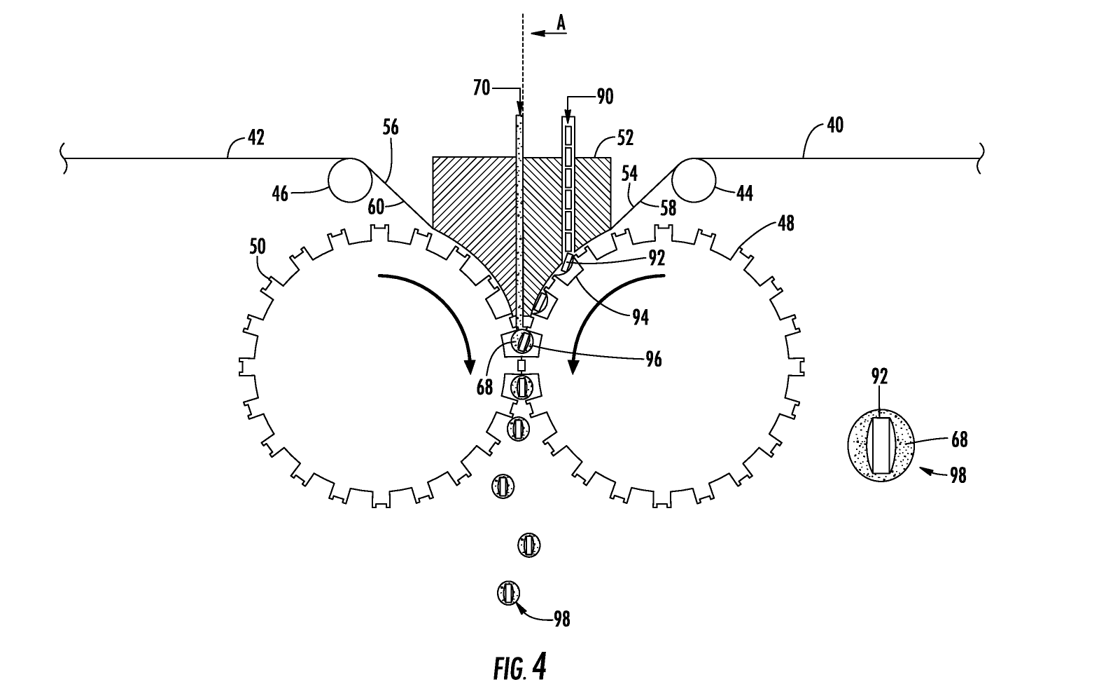

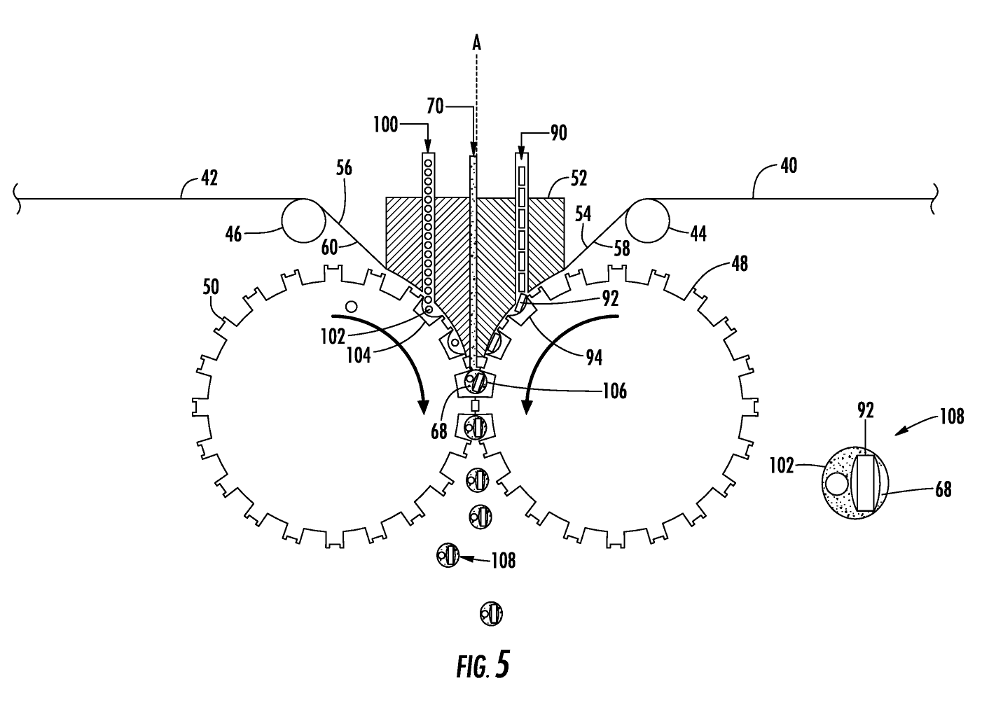

FIGS. 4 and 5 illustrate alternative embodiments of producing the soft elastic capsules described herein. In FIG. 4, a tablet hopper 90 feeds a compressed tablet 92 into a die cavity 94 located on encapsulation die 48. The tablet 92 is released from the tablet hopper 90 and positioned onto the surface 54 of the film 40. The tablet hopper 90 is located on an end of the encapsulation wedge 52 distal to the center plane A of the apparatus. The positioning of the tablet 92 forms an indentation in the film 40 into the die cavity 94. The tackiness of the surface 54 and the encapsulation wedge 52 keep the tablet 92 on the film 40. As a result, the tablet 92 is provided in the partially closed capsule 96 formed by the film 40 and the film 42. The liquid or semisolid fill 68 can be fed from channel 70 as described above into the partially closed capsule 96. The partially closed capsule 96 can then be sealed as described above to form a soft capsule 98 containing the tablets 92 and the fill 68.

As shown in FIG. 5, a second tablet hopper 100 can optionally be provided in the same manner as tablet hopper 90 to provide a second tablet 102. The second compressed tablet 102 can be situated in a die cavity 104 located on encapsulation die 50. The tablet 102 is released from the tablet hopper 100 and positioned onto the surface 56 of the film 42. As shown in FIG. 5, the tablet hopper 100 can be located on an end of the encapsulation wedge 52 opposite the tablet hopper 90. As with the tablet 92, the positioning of the second tablet 102 forms an indentation in the film 42 into the die cavity 104 and is then advanced to be provided in the partially closed capsule 106 formed by the film 40 and the film 42. The tablet 92 and the liquid or semisolid fill 68 can also be fed as described above into the partially closed capsule 106. The partially closed capsule 106 can then be sealed as described above to form a soft capsule 108 containing tablets 92 and 102 and the fill 68.

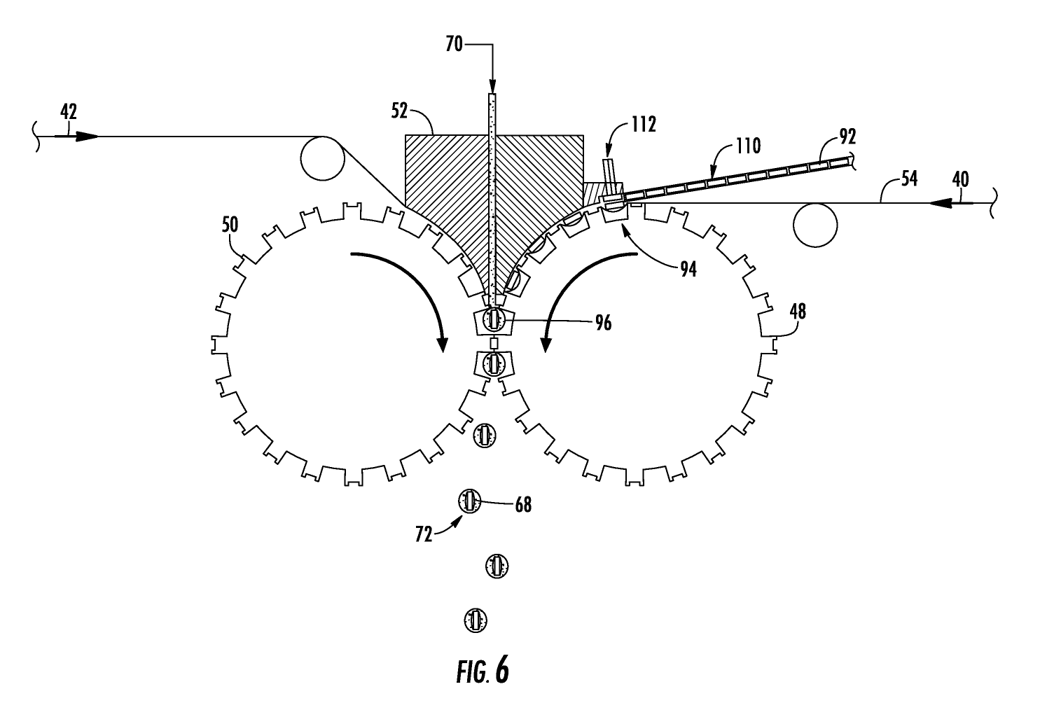

Optionally, the tablet 92 can be dispensed from a tablet hopper 110, located adjacent to the encapsulation wedge 52, onto the film 40 as illustrated in FIG. 6. In FIG. 6, the tablet 92 is released from the tablet hopper 110 and positioned on the surface 54 of the film 40. The die 48 rotates to the position of the ejector pin 112 and the tablet 92 is positioned into the die cavity 94 located on die 48. As described above, the liquid or semisolid fill 68 can be fed from channel 70, through the encapsulation wedge 52, into the partially closed capsule 96 and sealed to form the soft capsule 72. The soft capsule 72 is released from the encapsulation dies 48 and 50 located on the encapsulation dies.

In some embodiments, the strength of the capsule shell can be tested using methods known to those of skill in this art. For example, the strength of the gelatin used to form a soft capsule as described herein can be determined by measuring the Bloom strength. The Bloom strength test determines the force (e.g., in grams) needed by a probe to deflect the surface of the gel without breaking it. The cylindrical probe used for the test has a diameter of about 0.5 inch and the deformation of the gel tested can be about 4 mm. The result is expressed in Bloom (grams). Gels suitable for use as the capsule shell have a Bloom strength between 30 and 300 Bloom (e.g., between 100 and 200 Bloom).

The examples below are intended to further illustrate certain aspects of the methods and compositions described herein, and are not intended to limit the scope of the claims.

### EXAMPLES

### Capsule Shell Compositions

TABLE 1
Capsule Shell Composition 1

| Component | Component % (weight) |
|---|---|
| Gelatin | 35 |
| Glycerin | 16 |
| Water | 46 |
| Pectin | 3 |
| Calcium Chloride Dihydrate | 0.006 |

The components listed above were combined and mixed to form Capsule Shell Composition 1, a film-forming polymer for use as a capsule shell. Capsule Shell Composition 1 includes pectin as a gastric-resistant polymer.

TABLE 2
Capsule Shell Composition 2

| Component | Component % (weight) |
|---|---|
| Gelatin | 28 |
| Glycerin | 18 |
| Water | 41 |
| EUDRAGIT L100 | 11 |
| Triethyl Citrate | 1 |

The components listed above were combined and mixed to form Capsule Shell Composition 2, a film-forming polymer for use as a capsule shell. Capsule Shell Composition 2 includes EUDRAGIT L 100, a gastric-resistant anionic copolymer based on methacrylic acid and methyl methacrylate (Evonik Industries; Essen, Germany).

### Soft Elastic Capsule Formulations

TABLE 3
Examples 1-4

| | Example 1 | Example 2 | Example 3 | Example 4 |
|---|---|---|---|---|
| Shell Type | Composition 1 | Composition 2 | Composition 1 | Composition 1 |
| Fill (mg) | Fish oil | Fish oil (1000 mg) | Diphenhydramine | Simethicone |
| | (1000 mg) | | (50 mg) | (110 mg) |
| Tablet (mg) | Enteric-coated | Atorvastatin | Loratadine | Loperamide |
| | aspirin (81 mg) | (10 mg) | (10 mg) | (2 mg) |

Examples 1-4 were prepared using a rotary die process as described herein. The capsule shells were prepared from either Capsule Shell Composition 1 or 2, as indicated in the table above. Example 1 contained a fish oil fill and an enteric-coated aspirin tablet. Example 2 contained a fish oil fill and an atorvastatin tablet. Example 3 contained a diphenhydramine fill and a loratadine tablet. Example 4 contained simethicone as the fill and loperamide as the tablet. The amounts of each of the components are shown in the table above. The chemical stability of the aspirin in the fish oil fill of Example 1 under different storage conditions over time was determined and is reported in Table 1 as a percentage of the active ingredient left as compared with the label claim. RH refers to relative humidity.

TABLE 4
Capsule Stability

| | 0 months | 2.6 months | 6 months |
|---|---|---|---|
| Ambient | — | 99.70% | — |
| 30° C./65% RH | 99.70% | 99.22% | 98.50% |
| 40° C./75% RH | 99.70% | 96.20% | 75.26% |

TABLE 5
Examples 5-9

| | Example 5 | Example 6 | Example 7 | Example 8 | Example 9 |
|---|---|---|---|---|---|
| Fill (mg) | Fish oil | Fish oil | Fish oil | Fish oil | Fish oil |
| | (1000 mg) | (1000 mg) | (1000 mg) | (1000 mg) | (1000 mg) |
| Tablet (mg) | Clopidogrel | Phytosterol | Coenzyme Q10 | Resveratrol | Bexarotene |
| | (75 mg) | (1000 mg) | (200 mg) | (20 mg) | (75 mg) |

TABLE 6
Examples 10-14

| | Example 10 | Example 11 | Example 12 | Example 13 | Example 14 |
|---|---|---|---|---|---|
| Fill (mg) | Cetirizine | Methysergide | Metformin | Lubiprostone | Metformin |
| | (10 mg) | (8 mg) | (500 mg) | (24 μg) | (500 mg) |
| Tablet (mg) | Pseudoephedrine | Bupropion | Pioglitazone | Oxycodone | Miglitol |
| | (120 mg) | (300 mg) | (30 mg) | (10 mg) | (50 mg) |

Examples 5-14 are prepared using a rotary die process as described herein. The capsule shells are prepared from either Capsule Shell Composition 1 or 2 (see Tables 1 and 2). The amounts of each of the components are shown in the table above.

The capsules and methods of the appended claims are not limited in scope by the specific capsules and methods described herein, which are intended as illustrations of a few aspects of the claims and any capsules and methods that are functionally equivalent are intended to fall within the scope of the claims. Various modifications of the capsules and methods in addition to those shown and described herein are intended to fall within the scope of the appended claims.

Further, while only certain representative capsules and method steps disclosed herein are specifically described, other combinations of the capsules and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed.

What is claimed is:

1. A soft elastic capsule, comprising:
an acid resistant, capsule shell that defines an encapsulated space having a predetermined volume;
a liquid or semisolid fill comprising a first active ingredient located within the encapsulated space; and
a first compressed tablet having a minimal dimension of 2 mm, being located within the encapsulated space, unanchored to the capsule shell, and surrounded by the fill, said tablet comprising a second active ingredient and being substantially insoluble in the fill.

2. The soft elastic capsule of claim 1, wherein the first compressed tablet has a minimal dimension of 5 mm.

3. The soft elastic capsule of claim 1, wherein the first compressed tablet has a maximum dimension of 16 mm.

4. The soft elastic capsule of claim 1, wherein the volume of the tablet is at least 25% smaller than the volume of the encapsulated space.

5. The soft elastic capsule of claim 1, wherein the first active ingredient is a pharmaceutically active ingredient dissolved in a pharmaceutically acceptable oil-based liquid vehicle and the second active ingredient is a pharmaceutically active ingredient.

6. The soft elastic capsule of claim 1, wherein the first active ingredient is a polyunsaturated fatty acid and the second active ingredient is acetylsalicylic acid.

7. The soft elastic capsule of claim 6, wherein the first active ingredient is an omega-3 fatty acid.

8. The soft elastic capsule of claim 1, wherein the first active ingredient is a polyunsaturated fatty acid and the second active ingredient is clopidogrel.

9. The soft elastic capsule of claim 1, wherein the first active ingredient is a polyunsaturated fatty acid and the second active ingredient is phytosterol, coenzyme Q10, or resveratrol.

10. The soft elastic capsule of claim 1, wherein the first active ingredient is a polyunsaturated fatty acid and the second active ingredient includes bexarotene, a statin, or a combination of these.

11. The soft elastic capsule of claim 1, wherein the first active ingredient is diphenhydramine and the second active ingredient is loratadine.

12. The soft elastic capsule of claim 1, wherein the first active ingredient is simethicone and the second active ingredient is loperamide.

13. The soft elastic capsule of claim 1, wherein the first active ingredient is an anti-allergy agent and the second active ingredient is pseudoephedrine.

14. The soft elastic capsule of claim 1, wherein the first active ingredient is a serotonin 5-HT1A partial agonist or a selective serotonin re-uptake inhibitor and the second active ingredient is bupropion.

15. The soft elastic capsule of claim 1, wherein the first active ingredient is metformin and the second active ingredient is miglitol, pioglitazone, or a combination of these.

16. The soft elastic capsule of claim 1, wherein the first active ingredient is lubiprostone and the second active ingredient is an opioid.

17. The soft elastic capsule of claim 1, wherein the capsule shell is formed of a film-forming natural polymer and the film-forming natural polymer includes gelatin.

18. The soft elastic capsule of claim 17, wherein the film-forming natural polymer is from about 20% to about 40% by weight of the capsule shell.

19. The soft elastic capsule of claim 1, wherein the capsule shell includes an enteric coating layer.

20. The soft elastic capsule of claim 1, wherein the capsule shell is further formed of a gastric-resistant natural polymer.

21. The soft elastic capsule of claim 20, wherein the gastric-resistant natural polymer includes pectin.

22. The soft elastic capsule of claim 20, wherein the gastric-resistant natural polymer includes alginate.

23. The soft elastic capsule of claim 20, wherein the concentration of the gastric-resistant natural polymer is from about 2% to about 10% by weight of the capsule shell.

24. The soft elastic capsule of claim 1, wherein the capsule shell is further formed of a gelling agent.

25. The soft elastic capsule of claim 24, wherein the gelling agent includes a divalent cation salt selected from the group consisting of calcium salts and magnesium salts.

26. The soft elastic capsule of claim 24, wherein the concentration of the gelling agent is less than about 2% by weight of the capsule shell.

27. The soft elastic capsule of claim 1, wherein the capsule shell comprises one or more plasticizers in an amount of from about 8% to about 40% by weight of the capsule shell.

28. The soft elastic capsule of claim 1, wherein the second active ingredient is incompatible with the first active ingredient.

29. A method of manufacturing a soft elastic capsule, comprising:
(a) forming a continuous first film comprising a film-forming polymer on a first rotating encapsulation die;
(b) forming a continuous second film comprising a film-forming polymer on a second rotating encapsulation die;
(c) rotating the first rotating encapsulation die and the second rotating encapsulation die in counter directions to contact the first film and second film and form a partially closed capsule;
(d) providing a first compressed tablet in the partially closed capsule;
(e) injecting a liquid or semisolid fill into the partially closed capsule;
(f) sealing the partially closed capsule to form a soft capsule; and
(g) drying and finishing the soft capsule.
